(19) United States
(12) Patent Application Publication
(10) Pub. No.: US 2013/0123469 A1
(43) Pub. Date: May 16, 2013

(54) FRACTIONATION OF PROTEINS AND LIPIDS FROM MICROALGAE

(71) Applicant: OLD DOMINION UNIVERSITY RESEARCH FOUNDATION, Norfolk, VA (US)

(72) Inventors: Sandeep Kumar, Virginia Beach, VA (US); Patrick G. Hatcher, Suffolk, VA (US)

(73) Assignee: OLD DOMINION UNIVERSITY RESEARCH FOUNDATION, Norfolk, VA (US)

(21) Appl. No.: 13/707,754

(22) Filed: Dec. 7, 2012

Related U.S. Application Data
(63) Continuation-in-part of application No. 13/096,016, filed on Apr. 28, 2011.
(60) Provisional application No. 61/568,858, filed on Dec. 9, 2011.

(51) Int. Cl. C07K 1/12

(52) U.S. Cl. CPC C07K 1/12
USPC 530/370

(57) ABSTRACT

Methods of fractionating proteins and lipids in algae are provided. The methods can extract peptides and amino acids from algae, can produce components used in food or fertilizer, and can improve algal biomass feed for biofuel production, as non-limiting examples. One embodiment of a method as disclosed herein comprises providing a feed material of algae saturated with water to a reactor, bringing the water saturating the algae to a subcritical temperature within the reactor and separating a reactor effluent into solids and liquid.

### CROSS REFERENCE TO RELATED APPLICATION

This application claims priority to U.S. Provisional Patent Application Ser. No. 61/568,858, filed on Dec. 9, 2011, and is a continuation-in-part of U.S. patent application Ser. No. 13/096,016, filed on Apr. 28, 2011, both incorporated herein by reference in their entirety.

### TECHNICAL FIELD

The disclosure herein relates in general to the fractionation of lipids and co-products such as protein flash hydrolysis of the microalgae.

### BACKGROUND

The recent emphasis on finding alternative energy sources to fuel the energy needs of the United States and the world is leading to an accelerated search for new sources of fuel. Producing fuel from biomass is an important focus of many alternative energy strategies. Microalgae are considered by those skilled in the art as one of the most promising feed stocks for biofuels. Interest in algae as a possible source of fuel has soared in recent years because of associated advantages that include, but are not limited to, (1) removing carbon dioxide from the atmosphere (2) non-competition with agricultural crops and (3) potential for greater gallon per acre biofuel production than currently used crops.

However, there are major challenges in accomplishing the objective of cost competitive biofuel production from microalgae, including for example, the removal of water and nitrogen from the microalgae during processing. When using microalgae as feedstock with conventional methods, energy required for the dewatering process may account for more than 75% of the total energy consumption. For example, typical thermal dryers use significantly energy per kilogram of evaporated water (3.3-3.9 MJ/kg). The drying steps also lead to large parasitic energy losses that can consume much of the energy content of the biomass.

Algal proteins and carbohydrates are conventionally extracted by classical methods and by employing enzymatic degradation procedures. The classical methods use alkali hydrolysis followed by acid precipitation for protein extraction. The process efficiency is typically limited by the linkages between polysaccharides and proteins. Protein yield is typically low due to the degradation at high pH and undesirable toxic products are produced. Concentrated alkali causes the breakdown of protein and other valuable compounds. Also, this classical process is associated with serious economic and environmental constraints due to the heavy use of chemicals. The enzymatic extraction processes are milder than the alkali hydrolysis and produce no toxic chemicals. However, slow conversion rates and high enzyme costs make the process uneconomical.

### SUMMARY

Disclosed herein are methods of fractionating proteins and lipids in algae. The methods can extract peptides and amino acids from algae, can produce components used in food or fertilizer, and can improve algal biomass feed for biofuel production, as non-limiting examples. One embodiment of a method as disclosed herein comprises providing a feed material of algae saturated with water to a reactor, bringing the water saturating the algae to a subcritical temperature within the reactor and separating a reactor effluent into solids and liquid.

In one aspect, the feed material comprises an algae source and a water source mixed prior to entering the reactor. In one aspect, the subcritical temperature can be between 200° C. and 350° C. In one aspect, the reactor is a continuous feed reactor and a residence time of within the reactor is less than two minutes. The residence time can be, for example, less than or equal to ten seconds.

In other aspects of the embodiments, the algae saturated with water can have a solids load of between 0.01 and 7.5 dry weight percent. The liquid can be recycled for use in algae cultivation. The subcritical temperature in combination with the residence time is configured to extract at least 60 percent of a total nitrogen content in the algae from the solids into the liquid. The liquid contains peptides and free amino acids. The subcritical temperature in combination with the residence time can be configured to optimize the lipid concentration in the solids.

Another embodiment of a method of fractionating proteins from algae disclosed herein comprises feeding to a reactor an algal biomass, providing a solvent consisting essentially of water to the reactor, using the solvent as a reaction medium in the reactor by bringing the solvent to a subcritical temperature within the reactor and separating a reactor effluent into solids and liquid. Feeding the algal biomass and providing the solvent can occur in a single feed stream to the reactor.

The subcritical temperature of the reaction medium can be selected to increase nitrogen extraction from the algal biomass to the liquid. For example, the subcritical temperature can be between 280° C. and 350° C. The subcritical temperature of the reaction medium can be selected to increase lipid concentration in the solids. For example, the subcritical temperature can be between 200° C. and 300° C.

### BRIEF DESCRIPTION OF THE DRAWINGS

The description herein makes reference to the accompanying drawings wherein like reference numerals refer to like parts throughout the several views, and wherein:

FIG. 1 is a schematic of a method of fractionating proteins and lipids from algae as disclosed herein;

FIG. 2 is a flow diagram of a method of fractionating proteins and lipids from algae as disclosed herein;

FIGS. 3A and 3B respectively show the amino acid profile of the liquid products prior to and after hydrolyzation of peptides and proteins obtained at a first set of reaction conditions;

FIGS. 4A and 4B respectively show the amino acid profile of the liquid products prior to and after hydrolyzation of peptides and proteins obtained at a second set of reaction conditions;

FIGS. 5A and 5B respectively show the amino acid profile of the liquid products prior to and after hydrolyzation of peptides and proteins obtained at a third set of reaction conditions;

FIGS. 6A and 6B respectively show the amino acid profile of the liquid products prior to and after hydrolyzation of peptides and proteins obtained at a fourth set of reaction conditions;

FIGS. 7A and 7B respectively show the amino acid profile of the liquid products prior to and after hydrolyzation of peptides and proteins obtained at a fifth set of reaction conditions;

FIG. 8 shows that extracted nitrogen from algae using a method described herein is primarily available as proteins, peptides, and amino acids;

FIG. 9 shows the nitrogen content remaining in the solids after treatment with a method disclosed herein;

FIG. 10 shows the carbon content in the solids after treatment with a method disclosed herein;

FIG. 11 shows the nitrogen balance of the liquids and solids after treatment with a method disclosed herein;

FIG. 12 shows the lipid recovery in the solids after treatment with a method disclosed herein;

FIG. 13 is pyrolysis gas chromatogram of the initial microalgae and of solid residues after treatment with a method disclosed herein, with protein derived compounds indicated with P, chlorophyll derived compounds with Pr and Ph and fatty acid with Fa; and

FIGS. 14A and 14B are images taken with a scanning electrode microscope of solids before and after treatment with a method disclosed herein.

### DETAILED DESCRIPTION

Microalgae (also referred to as algae or an algal biomass) have the potential of producing biomass at rate orders of magnitude higher than traditional crops. The major components of microalgae are protein, carbohydrates and lipids. When used to produce biofuels, microalgae protein fractionation is not efficient. Including protein extraction using subcritical water hydrolysis from microalgae before converting the microalgae to biofuels can improve protein fractionation, producing bioproducts from wet algae without the need of any chemical.

Microalgae are primarily comprised of varying proportions of proteins, carbohydrates, lipids, and mineral matter. The percentage of each of these components varies depending upon the species of algae. Table 1 shows the general composition of organic components in strains of different microalgae.

TABLE 1
General composition of different algae (% of dry matter).

| Alga | Protein | Carbohydrates | Lipids |
|---|---|---|---|
| Chlamydomonas rheinhardii | 48 | 17 | 21 |
| Chlorella vulgaris | 51-58 | 12-17 | 14-22 |
| Euglena gracilis | 39-61 | 14-18 | 14-20 |
| Porphyridium cruentum | 28-39 | 40-57 | 9-14 |
| Scenedesmus obliquus | 50-56 | 10-17 | 12-14 |
| Spirulina platensis | 46-63 | 8-14 | 4-9 |

The high lipid content of many forms of algae makes algal biomass useful for biofuel production. The portion of biomass that is non-lipid can provide a high-value co-product such as animal feed or fertilizer that offsets the cost of converting the algae to fuels. Growing algae also removes nitrogen and phosphorus from water and consumes carbon dioxide.

Microalgae are one of the most promising feed stocks for biofuels. The biomass can be used for producing solid (biochar), liquid (biodiesel, liquid hydrocarbons, pyrolysis oil), and gaseous fuels (synthesis gas, methane, hydrogen). However, the attractive target is producing fungible fuels such as gasoline, diesel, and jet fuel.

It is not uncommon to have less than one gram of algae per liter of water. Therefore, a cost efficient harvesting and drying process is needed to produce a biomass suitable for oil recovery. Conversion processes such as that disclosed herein that can process wet biomass are highly desirable for reducing the energy intensive dewatering cost.

Microalgae are rich in proteins as shown in Table 1. The elemental composition (carbon, hydrogen, and oxygen) of microalgae is similar to other cellulosic biomass but differs in nitrogen content. Apart from water, sunlight, and carbon dioxide, nitrogen and phosphorous are the primary nutrients that are required to grow microalgae. Based on the average elemental composition, microalgae can be represented by a general formula such as CH_{1.7}O_{0.4}N_{0.15}P_{0.0094}. The nitrogen content varies between 4 to 8 wt % of the dry biomass depending upon the physiological state and nutrient limitation condition of microalgae. Due to the high nitrogen content, NOx emission and losses of nitrogen fertilizer are a matter of great concern in addition to the high moisture content if whole microalgae are processed for biofuel.

The organically bound nitrogen converts to ammonia in a reducing atmosphere and to NOx in a combustion/oxidizing atmosphere during the biofuels conversion process. In biogas production, high nitrogen contents lead to ammonia toxicity during the anaerobic digestion process. Also, high nitrogen contents are reported to inhibit the digestion of algal biomass. Similarly, presence of nitrogen in biomass will cause the formation of NOx compounds during the gasification process which is conducted under a limited supply of oxygen. NOx is a greenhouse gas and heavily regulated environmental pollutant.

Organic nitrogen is mainly present in the proteins of microalgae. Accordingly, extracting this high value protein can make production of biofuels from microalgae both economic and sustainable. The major emphasis of the methods disclosed herein is to maximize extraction of the non-fuel components and maximize recycle of nutrients.

More than 50,000 species of microalgae are reported to exist. Algal biomass composition varies depending upon the species as shown in Table 1. One challenge addressed herein involves developing a process that can tolerate the complex compositions found within the different species of algae. To make biofuels cost competitive, processes for the 100% utilization of algal biomass components are needed.

The methods disclosed herein comprise subcritical water extraction of protein and carbohydrates utilizing the tunable transport and solvent properties of subcritical water (critical point: 374° C., 221 bar) for converting biomass to high energy density fuels and functional materials. Sub-critical water is a non-toxic, environmentally benign, inexpensive and tunable reaction medium for conducting ionic/free radical reactions.

Water has a relatively high critical point because of the strong interaction between the molecules and hydrogen bond. Liquid water below the critical point is referred to as subcritical water, whereas water above the critical point is called supercritical water. The density and dielectric constant of the water medium play major roles in solubilizing different compounds. Water at ambient conditions (25° C. and 0.1 MPa) is a good solvent for electrolytes because of its high dielectric constant, whereas most organic matter is sparingly soluble. As water is heated, the H-bonding starts weakening, allowing dissociation of water into acidic hydronium ions (H₃O⁺) and basic hydroxide ions (OH⁻). The structure of water changes significantly near the critical point because of the breakage of infinite networks of hydrogen bonds and water exists as separate clusters with a chain structure. In fact, the dielectric constant of water decreases considerably near the critical point, which causes a change in the dynamic viscosity and also increases the self-diffusion coefficient of water. The physiochemical properties of water, such as viscosity, ion product, density, and heat capacity, also change dramatically in the supercritical region with only a small change in the temperature or pressure, resulting in a substantial increase in the rates of chemical reactions. The unusual dielectric behavior and transport properties of water are significantly different than those of ambient water.

Subcritical water, also termed “hydrothermal media”, can be broadly defined as a water-rich phase above the boiling point. Subcritical water offers several advantages over other biofuels production methods. Some of the major benefits are (i) high energy and separation efficiency (water remains in the liquid phase and the phase change is avoided), (ii) versatility of chemistry (solid, liquid and gaseous fuels), (iii) reduced mass transfer resistance in hydrothermal conditions, (iv) ability to use mixed feedstock as well as wet biomass, and (iv) products are completely sterilized with respect to any pathogens including biotoxins, bacteria or viruses. The technology can be applied to produce solid (biochar), liquid (bioethanol, biocrude), and gaseous (methane, synthesis gas, hydrogen) fuels depending on the processing conditions. The substantial changes in the physical and chemical properties of water in the vicinity of its critical point can be utilized advantageously for converting lignocellulosic biomass/algae to desired biofuels.

To heat water from 25° C. to steam at 250° C. at 0.1 MPa (1 atm) requires 2869 kJ/kg of energy. To heat water at 25° C. to subcritical water at 250° C. at 5 MPa requires only 976 kJ/kg of energy. It is also possible to recover much of the heat from subcritical water. Therefore, the energy use for subcritical water extraction is less than one sixth the energy used for steam distillation. This decreased need for energy also means that the energy contained in the subcritical water is insufficient to vaporize the water on decompression.

In subcritical water processing, water is maintained in the liquid phase with the use of pressure greater than the vapor pressure of water at the reaction temperature. Thus energy (latent heat of vaporization of water 2.26 MJ/kg) required for the phase change of water from liquid to vapor phase is avoided. Due to the use of high pressure, cost is attributed to the equipment necessary to operate at the operating pressure. However, the energy needed for pumping the feed at high pressure is significantly lower than the energy needed to heat the reactor to the reaction temperature.

Because microalgae are a nutrient rich feedstock and a natural source of antioxidants and antimicrobial compounds, the methods herein combine a biofuels process with the production of useful co-products developed by extracting and retaining the functionality of cell components such as proteins, carbohydrates, ω-3 fatty acids, pigments, and vitamins.

Microalgae are relatively small and protected, in many cases, by a thick cell wall. The resistance of the algal cell wall to microbial attack is generally attributed to the discrete structural entities and resistance of cell walls to decompose.

Some groups of green algae, eustigmatophytes and dinoflagellate are known to metabolize single or multi-layers of protective outer wall that is composed of an aliphatic polyethylene biopolymer called algaenan. Algaenan is pyrolytically convertible to hydrocarbons, as disclosed in U.S. Pat. No. 8,080,679 issued Dec. 20, 2011. This protective algaenan biopolymer is a recalcitrant material that is insoluble and non-hydrolyzable. It has been shown that the algaenan may be selectively preserved in sediments because of its recalcitrance and is thought to be converted into petroleum over geological time. Previous research has shown that one can simulate the process by which this algaenan converts to petroleum-like hydrocarbons using pyrolysis approaches. When pyrolyzed, algaenan produce a suite of hydrocarbons not unlike many petroleum hydrocarbons. These hydrocarbons are likely the precursors of kerogen in shales that yield paraffinic petroleums upon natural maturation.

Average algal biomass that contains this aliphatic protective outer-wall is composed of approximately 50% protein, 20% carbohydrates, 10% refractory biopolymer (algaenan), and 15% lipid. Typically, very harsh conditions (e.g. mechanical and/or chemical extraction) are required to break the cell walls for extracting the bioactive compounds. Such processes generally affect the functionality of other cell compounds, like proteins. The methods disclosed herein use subcritical water based technology to fractionate microalgae components without affecting functionality.

In the subcritical region, the ionization constant (Kw) of water increases with temperature—about three orders of magnitude higher than that of ambient water—and the dielectric constant (∈) of water drops from 80 to 20. A low dielectric constant allows subcritical water to dissolve organic compounds, while a high ionization constant allows subcritical water to provide an acidic medium for the hydrolysis reactions. These ionic reactions can be dominant because of the liquid-like properties of subcritical water. Moreover, the physical properties of water, such as viscosity, density, dielectric constant and ionic product, can be tuned by small changes in pressure and/or temperature within subcritical region.

The methods disclosed herein can disrupt the cell walls of the algae without changing the functionality of other cell components. This can increase the accessibility of protein in the algae to the water molecules. The algal cell walls, in general, are organized in a conventional framework. The basic framework is highly polymeric. Interspersed within are lower molecular weight polymers and oligomers (often gel like fibers) and inorganic and non-monomeric compounds. The solvent properties of subcritical water can solubilize organics and disrupt the cell wall. This will lead to the hydrolysis of the amorphous and water-soluble components of algae at the reaction conditions. The increased accessibility of the material within the cell wall to water molecules enhances the extraction of proteins and other cell components.

Consequently, the processes disclosed herein will (1) enable the extraction high value proteins; and (2) reduce the nitrogen content and water content before the residual algal biomass is used for biofuels application. As mentioned above, thermochemical conversion to fuels without protein removal can introduce unwanted and problematic nitrogenous species. Microalgae contain very high levels of protein presently untapped as a resource. Algae proteins can be used, for example, as a supplement to the basic diet and to food products.

Reactions in subcritical water medium are typically very fast (on the order of few minutes or seconds) with water as the reactant leading to hydrolysis and rapid degradation of the polymeric structure of biomass. The hydrolysis of biopolymers in subcritical water is sensitive to residence time. In general, high conversion rates to desired products can be achieved at very short residence times (the order of seconds to few minutes).

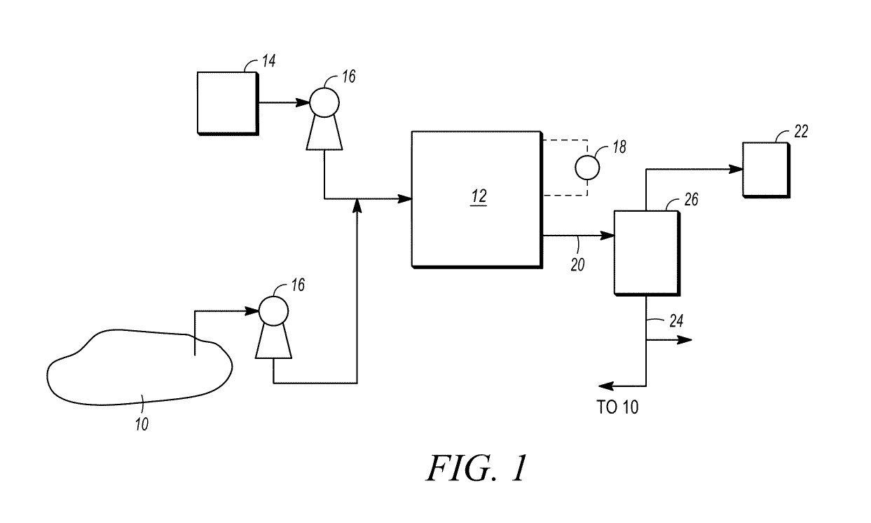

FIG. 1 is a schematic of one of the methods disclosed herein using high moisture content algal biomass as a feed stock and using water as a reactant as well as a reaction medium. A novel reactor design focuses on reaction engineering concepts to design a continuous flow reactor with a tunable residence time (in the order of seconds to a few minutes) to capitalizes on the difference in the hydrolysis kinetics of the microalgae components. The design of the continuous flow reactor avoids the preheating time encountered in batch and semi-batch reactors.

An algal biomass from an algae source 10, such as a pond, is fed to a reactor 12 in combination with additional water from a water source 14. The algal biomass and the additional water are each fed to the reactor with pumps 16. Both flow rates of the algal biomass and the additional water can be independently adjusted according to the desired or required conditions. The solids load can be modified by adjusting the pumps' flow rate, with typical solid loads between about 0.01 to 7.5 dw %, or more particularly, between about 0.9 to 1.2 dw %. The reactor 12 has a temperature control system 18 that allows the temperature of the reaction to be accurately controlled. To obtain the temperatures of the subcritical region of water, the water source 14 can be preheated. The water source 14 can also be recycled from the reactor 12, thereby requiring no further preheating. The reactor can be operated under pressure, with the pressure determined based on economically obtaining the subcritical region of water in the reactor 12.

The variable feed rate and solids load provides wide flexibility with regard to the solids retention time in the reactor 12, which ranges from a few seconds up to a few minutes.

The residence time is calculated as follows:

$$t = \frac{V}{F ( \frac{{ρ}_{pump}}{{ρ}_{P 1 , T 1}} )}$$

Where:
V is reactor volume;
F is the combined volumetric flow rate of pumps 16;
ρ pump is the density of water at pump condition; and
ρ_{P1,T1} is the density of water at reactor condition (e.g., T₁ and P₁).

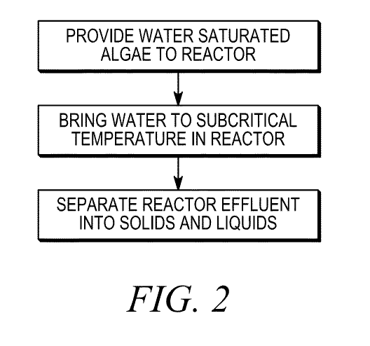

The reactor effluent 20 is separated into a solid phase 22 and a liquid phase 24 by any known means of separation 26, such as centrifugal force and filtration. The solids 22, rich with lipids and perhaps algaenan are an excellent feed material for biofuel processing. The liquid 24, high in nitrogen and therefore proteins can be used in various applications as discussed herein. The liquid 24 can also be recycled to provide nutrients to the algae cultivation. FIG. 2 is a flow diagram of a method disclosed herein.

The subcritical temperature at which the reactor operates can be varied, and is typically between about 200-350° C. Selectivity for the desired products can be achieved by tuning, which involves changing the operating conditions of one or more of residence time, pressure and temperature. The ionization constant of water increases with temperature below the critical point and this leads to hydrolysis, instead of pyrolysis. The intermediates from the depolymerization of biomass components show a high solubility in the water medium; hence reaction steps are mainly homogeneous. It is important to note that hydrolysis products are not the end products; rather they further convert to various degradation products if a longer residence time is allowed in the reactor. Therefore, optimization of residence time is the key to increase the selectivity of desired product and also suppress the degradation reactions.

The polymeric components of microalgae, namely carbohydrates, proteins, and lipids, have different depolymerization kinetics in the subcritical water medium. The methods herein utilize this difference in reaction kinetics of algae components to fractionate protein in liquid phase, and to optimize the retention of lipids along with algaenan in solid residue. Since carbohydrates are prone to be hydrolyzed along with protein, the liquid fractions after subcritical water hydrolysis are a mixture of sugars and water soluble protein.

The hydrolysis rate increases with reaction temperature for these polymers. The hydrolysis of polysaccharides starts above 180° C. in subcritical water within a residence time of seconds to a few minutes. The carbohydrates, such as hemicelluloses, starches, and amorphous cellulose, start depolymerizing to water soluble products in subcritical water above 180° C.

Peptide bonds of proteins exhibit much higher stability compared to the β-1,4- and β-1,6-glycosidic linkages in cellulose and starch, respectively. Protein hydrolyzes to amino acids which degrades subsequently and also forms free stable radical anions via the Maillard reaction of proteins and carbohydrates. Besides temperature, residence time is a crucial reaction parameter to stop degradation of the hydrolysis products.

Lipids are non-polar compounds. The reactions of lipids and water strongly depend on the phase behavior. The higher temperature causes increased solubility of fat and oils in subcritical water and ultimately they become completely soluble by the time water has reached its supercritical state. The hydrolysis of triacylglycerols (TAG) in subcritical water starts above 280° C. and conversion in excess of 95% can be achieved at 340° C. within a residence time of 12 min.

Algaenan is a non-hydrolyzable, insoluble biopolymer present in a variety of unicellular algae. It is recognized as an important component in kerogen and the largest organic carbon sink on the planet. Numerous chemical procedures have been proposed for the isolation of algaenan. They typically consist of treatment with a succession of organic solvents, acids, and bases, all of which lead to the removal of free lipids, carbohydrates and proteins. Algaenan has been isolated from the cell walls of chlorophycean algae from the genera Scenedesmus, Tetraedron, Chlorella, Botryococcus, and Haematococcus. U.S. patent application Ser. No. 13/096,016, filed on Apr. 28, 2011 and incorporated herein by reference, shows that subcritical water is capable of solubilizing the proteins, carbohydrates, and lipids present in the algae, while algaenan remains insoluble and recovered in the solid residue. This residue that contains mostly algaenan can be converted to hydrocarbon based fuels after 72 hours of subcritical water treatment at 360° C.

Examples of the methods disclosed herein follow. A continuous flow system was set using two high pressure pumps; one to pump only water and the second to indirectly pump algae slurry (Scenedesmus sp.) into a reactor inside a furnace.

In the first example, the reaction occurred at 205° C., the flow rate for both pumps was set so the solids retention time was approximately 10 seconds; and the solids load rate was approximately 1 dry wt %. The reaction was run for 25 minutes, followed by five minutes in which only water was fed to the reactor. This was done in order to collect the maximum amount of algae hydrolyzate and flush the system from possible biomass leftovers. After the 30 minutes just clear water was obtained indicating that all the biomass was flushed out of the system.

The hydrolyzate as collected contained both liquid and solid products; to separate them centrifugation and filtration was used.

TABLE 2
Example 1 solid and liquid product characteristics

| Solid Product | |
|---|---|
| Percentage solids recovered | 52.1% |
| % C (elemental analysis) | 54.7% |
| % N (elemental analysis) | 8.66% |
| Lipid content | 34% |
| Liquid Product | |
| Total Nitrogen (TN extracted %) | 27.4% |
| Total Organic Carbon (TOC extracted %) | 27.99% |
| pH | 6.5 |
| % of carbon as sugars (DNS method) | 0.66% |
| % of nitrogen as proteins (Lowry's method) | 30.23% |

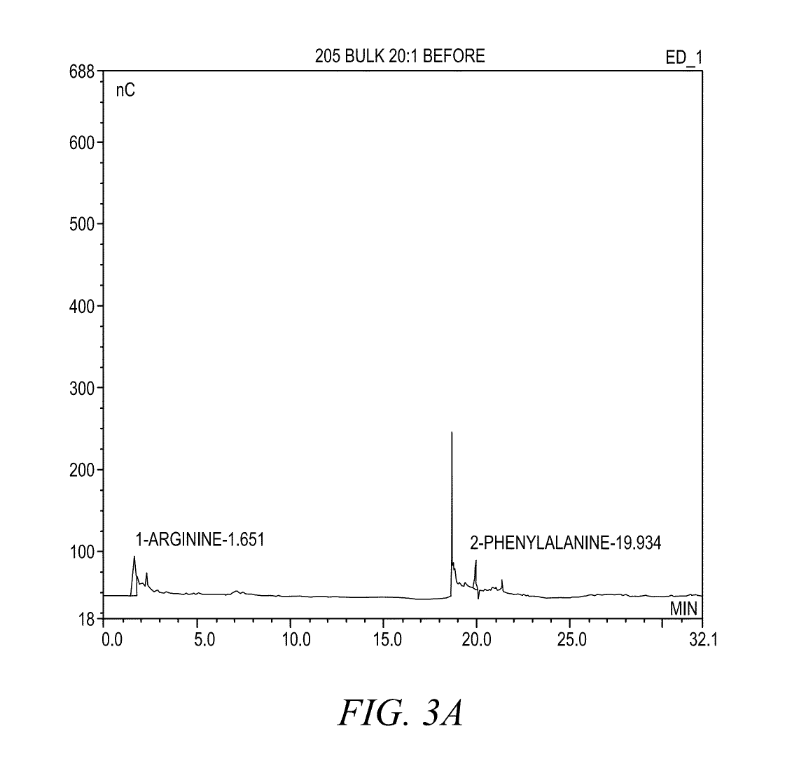

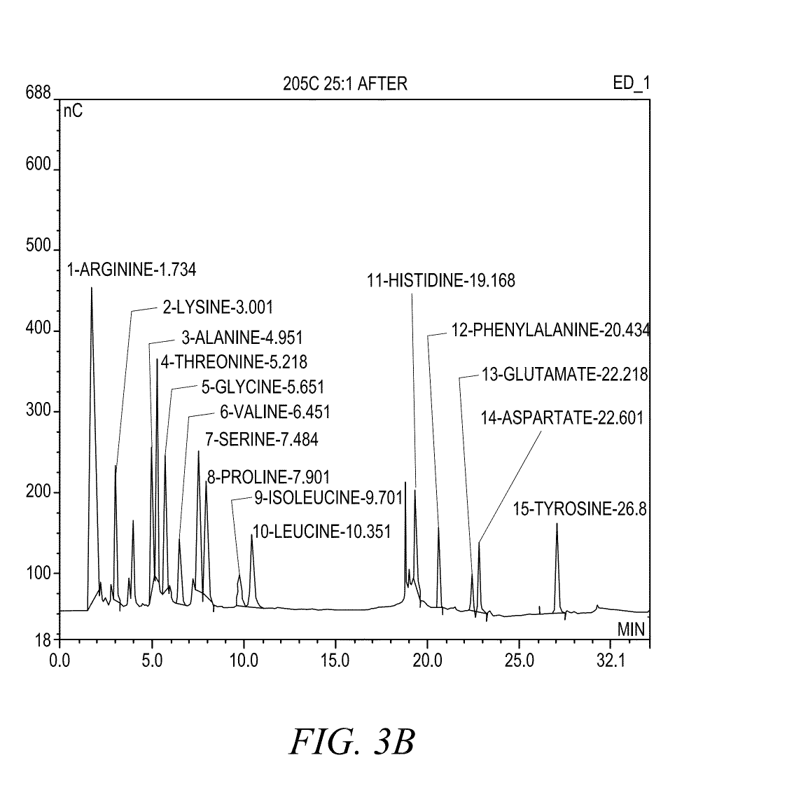

The chromatograms in FIGS. 3A and 3B show the amino acid profile of the liquid products. FIG. 3A shows almost no identifiable individual amino acids, while FIG. 3B corresponds to the same sample after being treated with diluted hydrochloric acid to further hydrolyze proteins and peptides and obtain free amino acids. This indicates that the majority of the hydrolyzed proteins are present in the forms of soluble peptides.

In the second example, the reaction occurred at 240° C., the flow rate for both pumps was set so the solids retention time was approximately 10 seconds; and the solids load rate was approximately 1 dry wt %. The reaction was run for 25 minutes, followed by five minutes in which only water was fed to the reactor. This was done in order to collect the maximum amount of algae hydrolyzate and flush the system from possible biomass leftovers. After the 30 minutes just clear water was obtained indicating that all the biomass was flushed out of the system.

The hydrolyzate as collected contained both liquid and solid products; to separate them centrifugation and filtration was used.

TABLE 3
Example 2 solid and liquid product characteristics

| Solid Product | |
|---|---|
| Percentage recovered | 38.48% |
| % C | 59.5% |
| % N | 8.05% |
| Lipid content | 61% |
| Liquid Product | |
| Total Nitrogen (TN extracted %) | 51.14% |
| Total Organic Carbon (TOC extracted %) | 46.9% |
| pH | 6.5 |
| % of carbon as sugars (DNS method) | 1.47% |
| % of nitrogen as proteins (Lowry's method) | 61.17% |

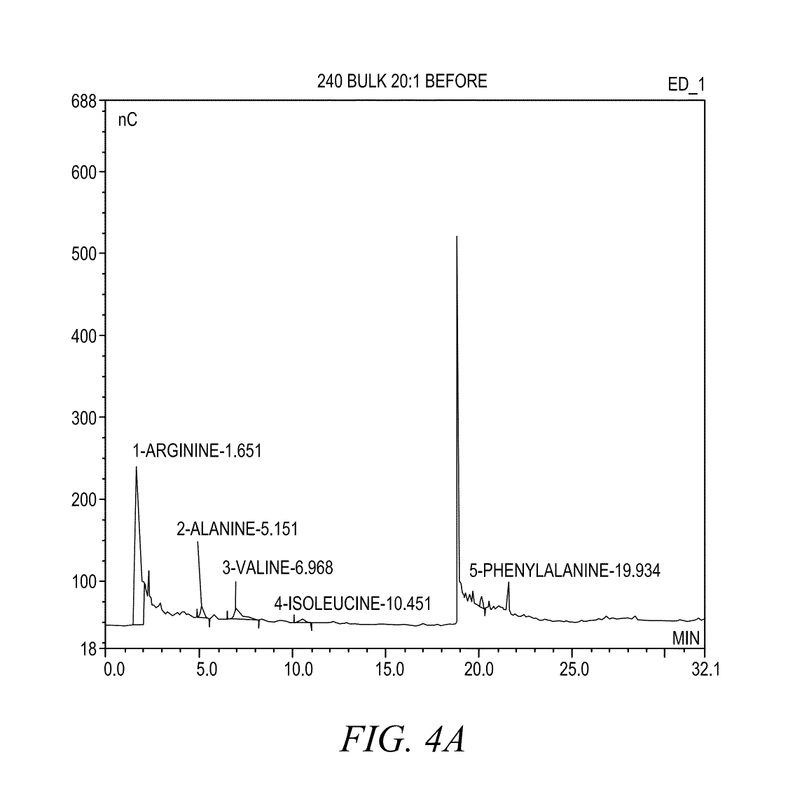

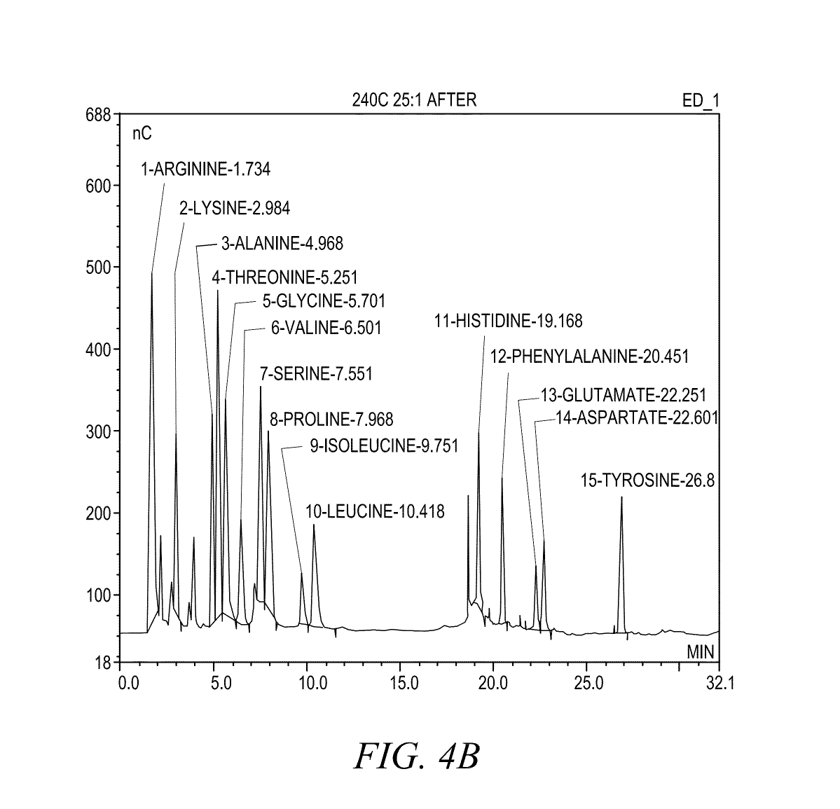

The chromatograms in FIGS. 4A and 4B show the amino acid profile of the liquid products. FIG. 4A shows almost no identifiable individual amino acids, while FIG. 4B corresponds to the same sample after being treated with diluted hydrochloric acid to further hydrolyze proteins and peptides and obtain free amino acids. This indicates that the majority of the hydrolyzed proteins are present in the forms of soluble peptides.

In the third example, the reaction occurred at 280° C., the flow rate for both pumps was set so the solids retention time was approximately 10 seconds; and the solids load rate was approximately 1 dry wt %. The reaction was run for 25 minutes, followed by five minutes in which only water was fed to the reactor. This was done in order to collect the maximum amount of algae hydrolyzate and flush the system from possible biomass leftovers. After the 30 minutes just clear water was obtained indicating that all the biomass was flushed out of the system.

The hydrolyzate as collected contained both liquid and solid products; to separate them centrifugation and filtration was used.

TABLE 4
Example 3 solid and liquid product characteristics

| Solid Product | |
|---|---|
| Percentage recovered | 30.03% |
| % C | 63.0% |
| % N | 7.56% |
| Lipid content | 44.8% |
| Liquid Product | |
| Total Nitrogen (TN extracted %) | 60.99% |
| Total Organic Carbon (TOC extracted %) | 54.52% |
| pH | 6.1 |
| % of carbon as sugars (DNS method) | 3.70% |
| % of nitrogen as proteins (Lowry's method) | 66.38% |

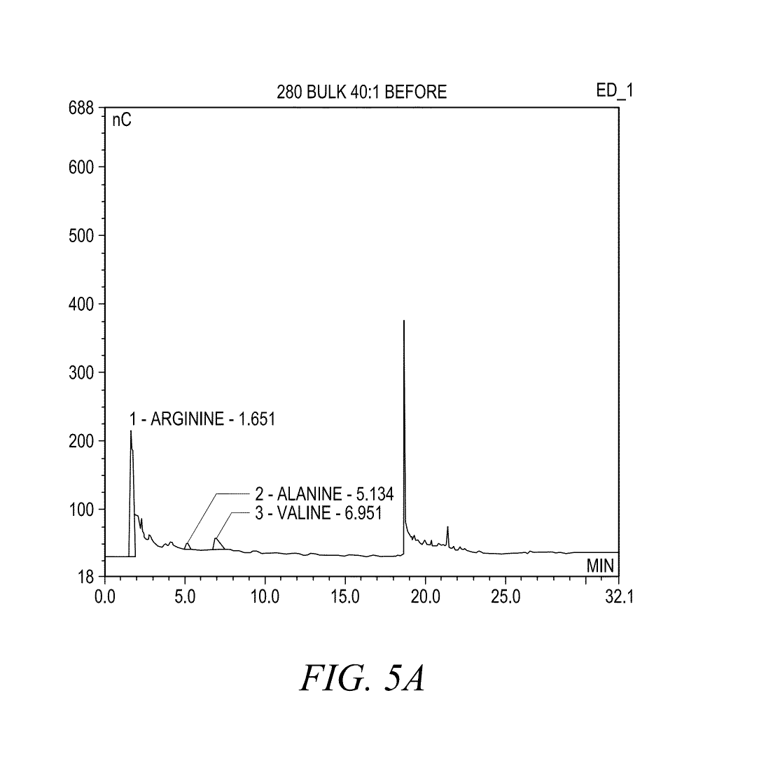

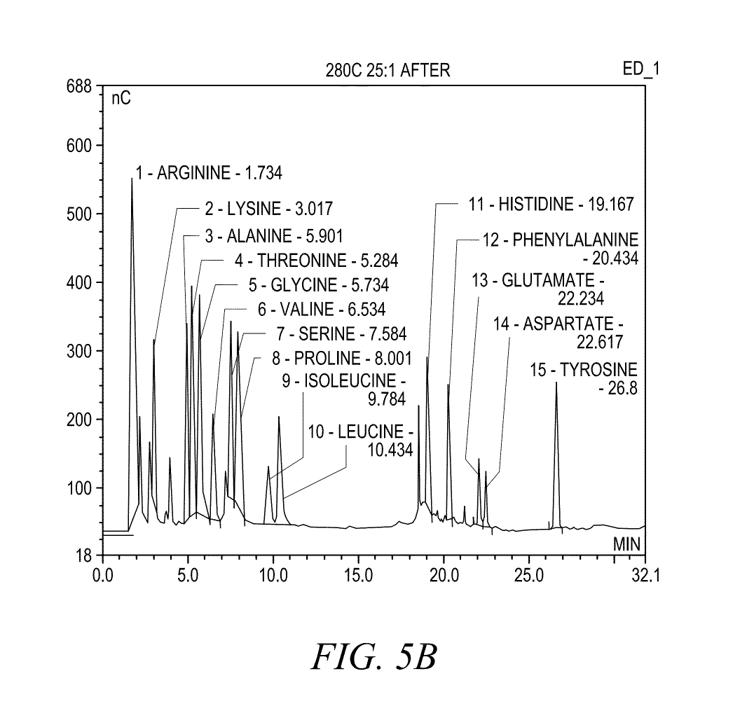

The chromatograms in FIGS. 5A and 5B show the amino acid profile of the liquid products. FIG. 5A shows almost no identifiable individual amino acids, while FIG. 5B corresponds to the same sample after being treated with diluted hydrochloric acid to further hydrolyze proteins and peptides and obtain free amino acids. This indicates that the majority of the hydrolyzed proteins are present in the forms of soluble peptides.

In the fourth example, the reaction occurred at 305° C., the flow rate for both pumps was set so the solids retention time was approximately 10 seconds; and the solids load rate was approximately 1 dry wt %. The reaction was run for 25 minutes, followed by five minutes in which only water was fed to the reactor. This was done in order to collect the maximum amount of algae hydrolyzate and flush the system from possible biomass leftovers. After the 30 minutes just clear water was obtained indicating that all the biomass was flushed out of the system.

The hydrolyzate as collected contained both liquid and solid products; to separate them centrifugation and filtration was used.

TABLE 5
Example 4 solid and liquid product characteristics

| Solid Product | |
|---|---|
| Percentage recovered | 26.74% |
| % C | 66.6% |
| % N | 6.59% |
| Lipid content | 71.8% |
| Liquid Product | |
| Total Nitrogen (TN extracted %) | 67.02% |
| Total Organic Carbon (TOC extracted %) | 56.67% |
| pH | 6.2 |
| % of carbon as sugars (DNS method) | 4.20% |
| % of nitrogen as proteins (Lowry's method) | 66.38% |

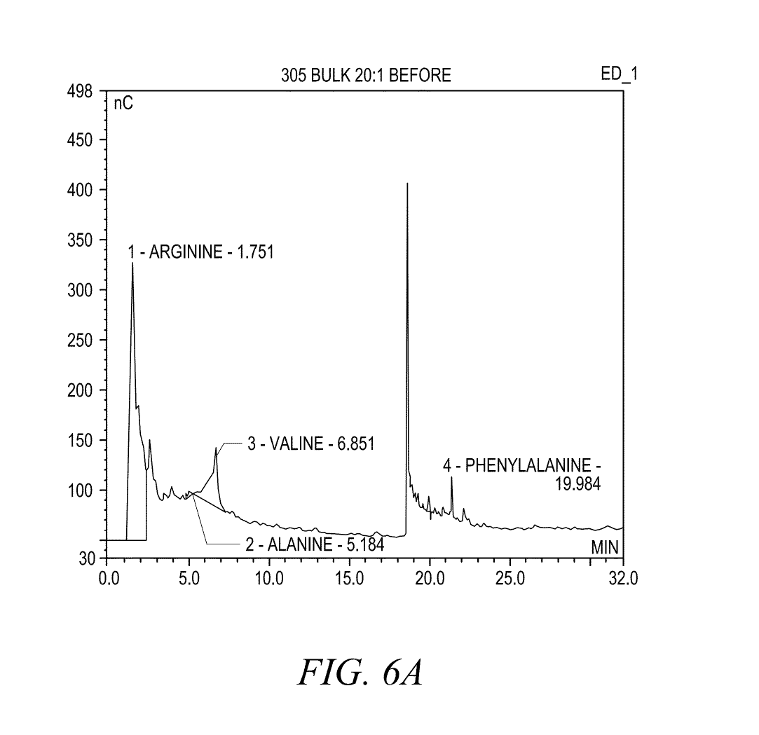

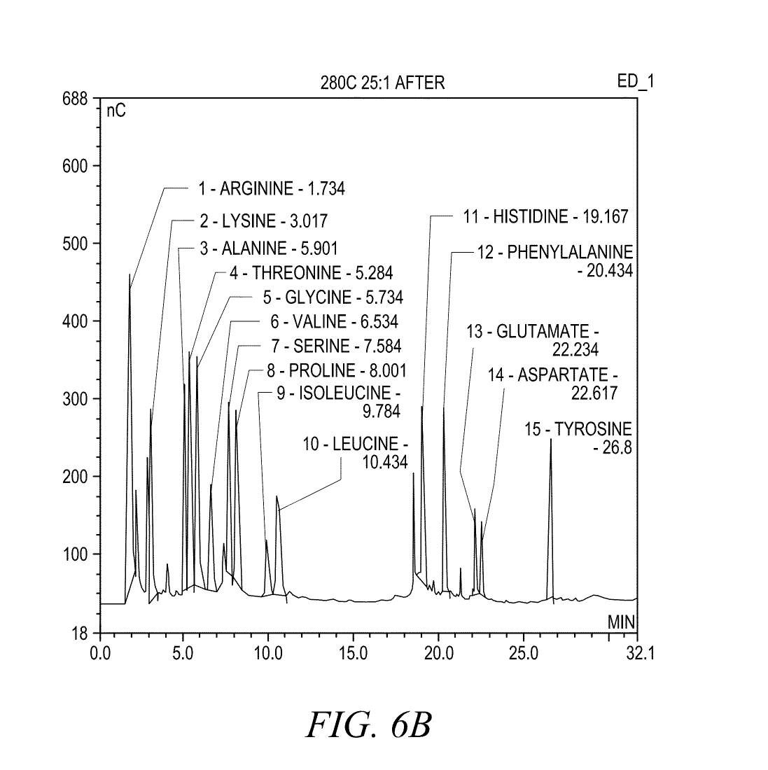

The chromatograms in FIGS. 6A and 6B show the amino acid profile of the liquid products. FIG. 6A shows almost no identifiable individual amino acids, while FIG. 6B corresponds to the same sample after being treated with diluted hydrochloric acid to further hydrolyze proteins and peptides and obtain free amino acids. This indicates that the majority of the hydrolyzed proteins are present in the forms of soluble peptides.

In the fifth example, the reaction occurred at 325° C., the flow rate for both pumps was set so the solids retention time was approximately 10 seconds; and the solids load rate was approximately 1 dry wt %. The reaction was run for 25 minutes, followed by five minutes in which only water was fed to the reactor. This was done in order to collect the maximum amount of algae hydrolyzate and flush the system from possible biomass leftovers. After the 30 minutes just clear water was obtained indicating that all the biomass was flushed out of the system.

The hydrolyzate as collected contained both liquid and solid products; to separate them centrifugation and filtration was used.

TABLE 6
Example 5 solid and liquid product characteristics

| Solid Product | |
|---|---|
| Percentage recovered | 24.04% |
| % C | 64.12% |
| % N | 5.99% |
| Lipid content | 67.2% |
| Liquid Product | |
| Total Nitrogen (TN extracted %) | 67.85% |
| Total Organic Carbon (TOC extracted %) | 58.31% |
| pH | 6.7 |
| % of carbon as sugars (DNS method) | 4.68% |
| % of nitrogen as proteins (Lowry's method) | 62.08% |

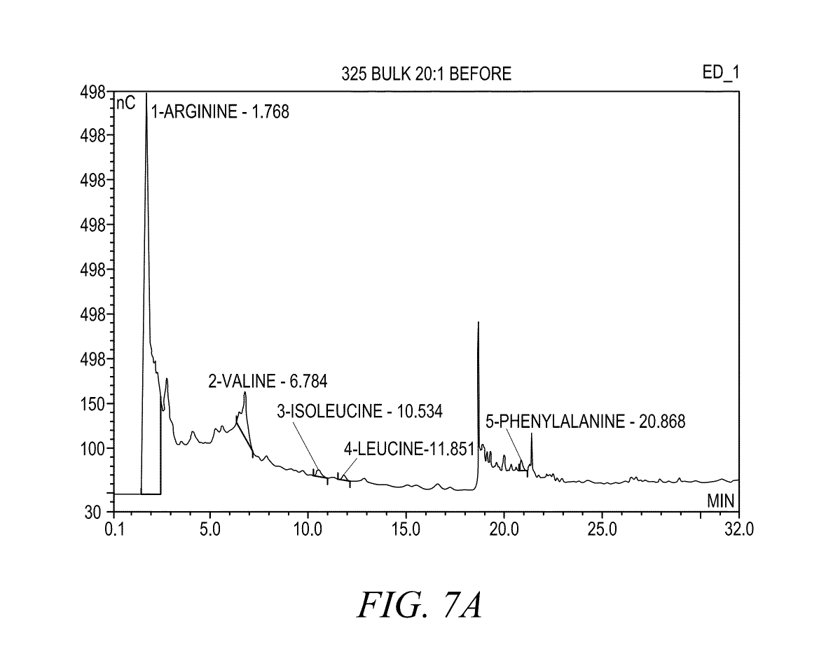

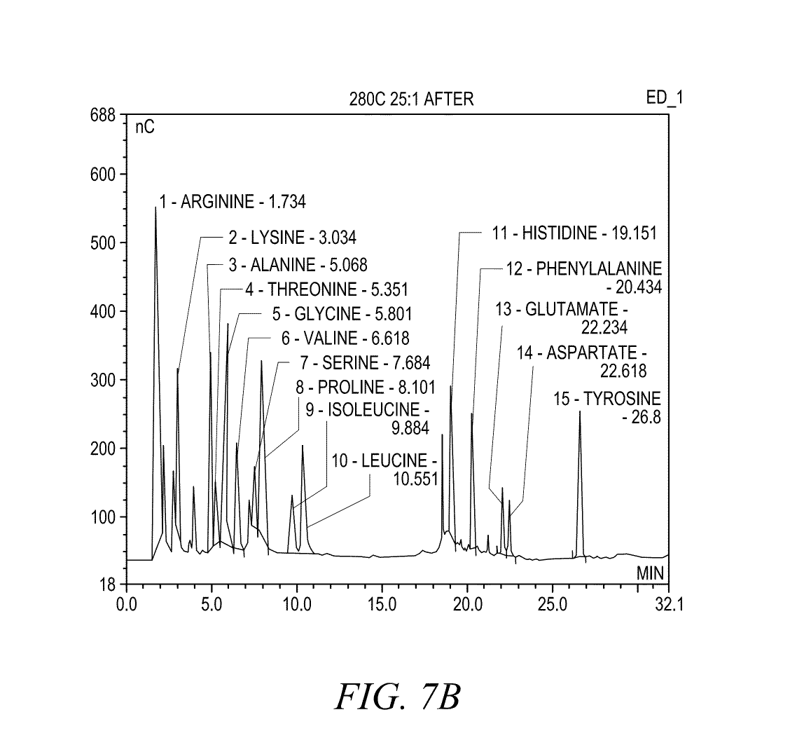

The chromatograms in FIGS. 7A and 7B show the amino acid profile of the liquid products. FIG. 7A shows almost no identifiable individual amino acids, while FIG. 7B corresponds to the same sample after being treated with diluted hydrochloric acid to further hydrolyze proteins and peptides and obtain free amino acids. This indicates that the majority of the hydrolyzed proteins are present in the forms of soluble peptides.

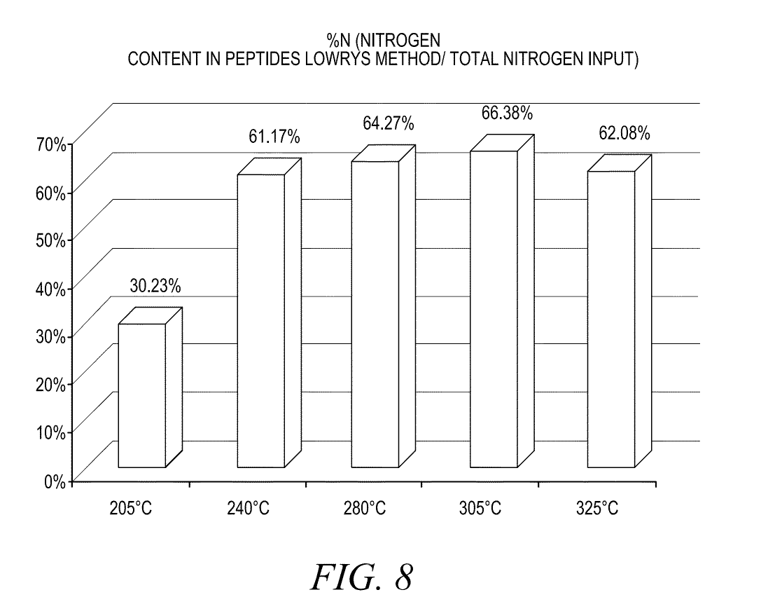

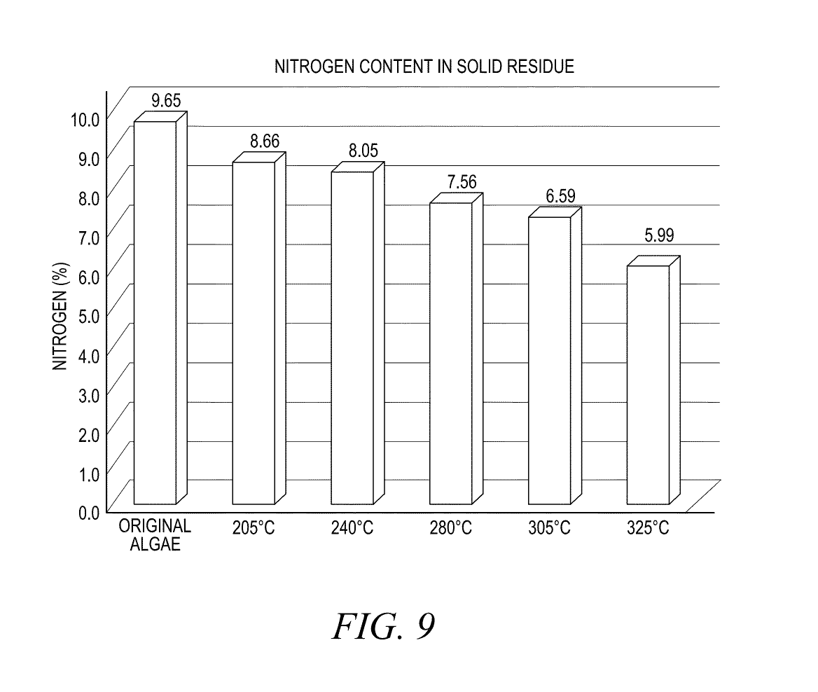

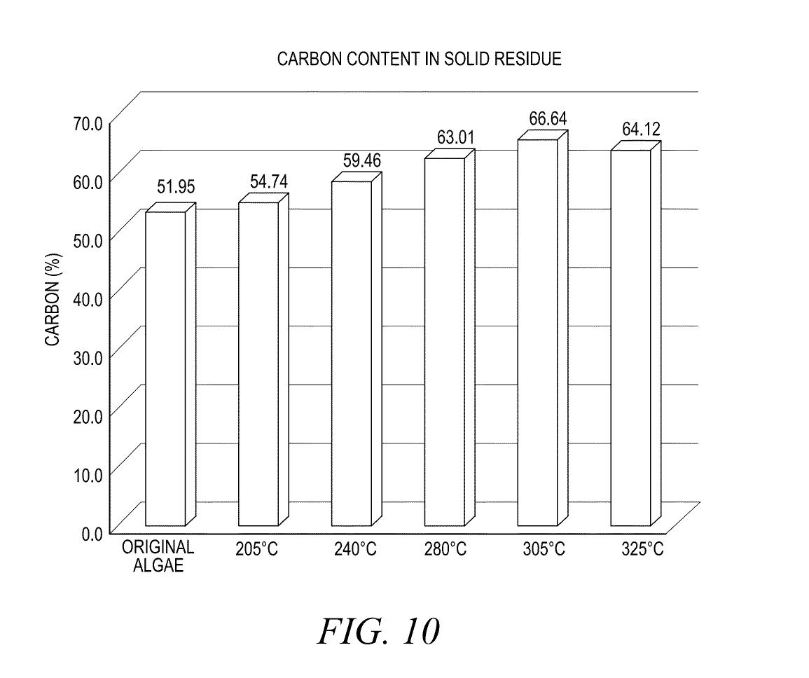

The overall results of examples one through five are detailed in FIGS. 8-14. In the liquid products, at as low as 240° C., more than 60% of the nitrogen is extracted. FIG. 8 shows that the extracted nitrogen from algae is mostly available as proteins, peptides and amino acids.

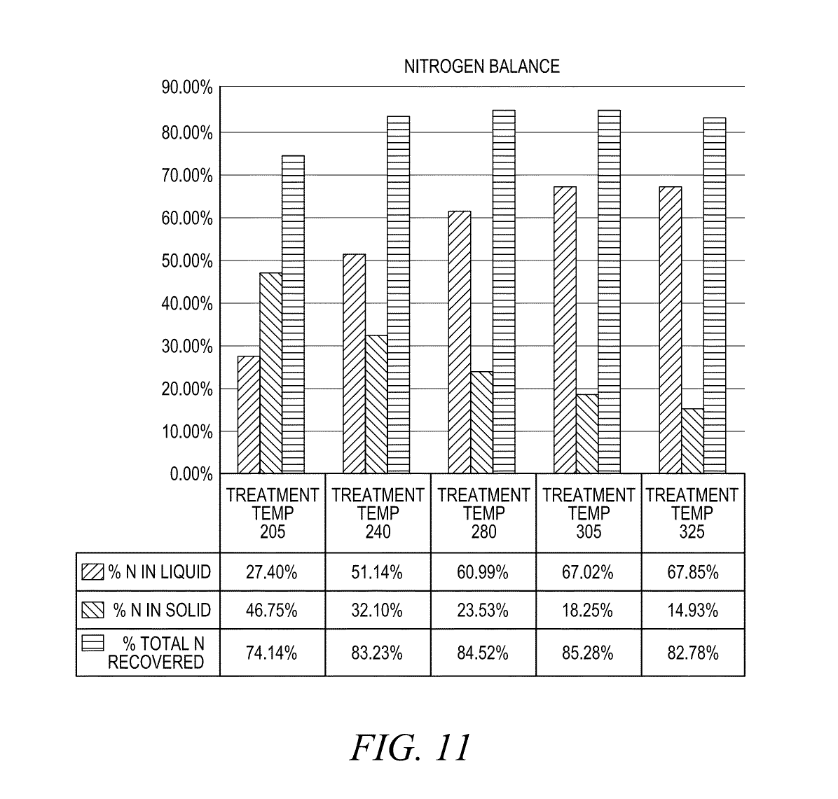

FIG. 11 clearly indicates that less than 25 wt % of nitrogen remains in the algal biomass after the flash hydrolysis as compared to the raw microalgae. Therefore, more than 75% of nitrogen present in the original microalgae was extracted at a temperature of 280° C. or above. This material balance does not account for the gaseous products.

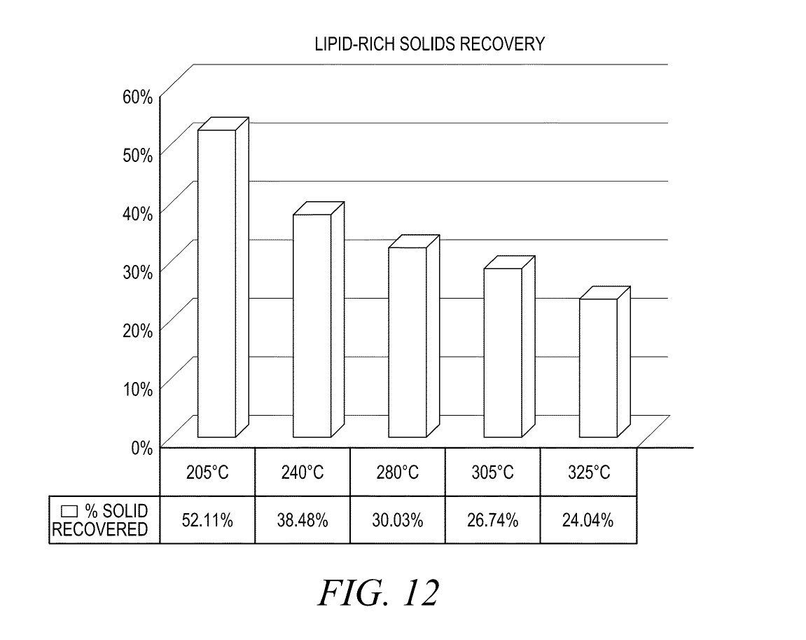

The solids were analyzed by nuclear magnetic resonance (NMR) and pyrolysis gas chromatography mass spectroscopy (pyr-GC-MS) techniques to conclude the presence of algaenan and lipids in solid samples. As shown in FIG. 12, the residual solids became richer in lipid content after the subcritical water extraction process.

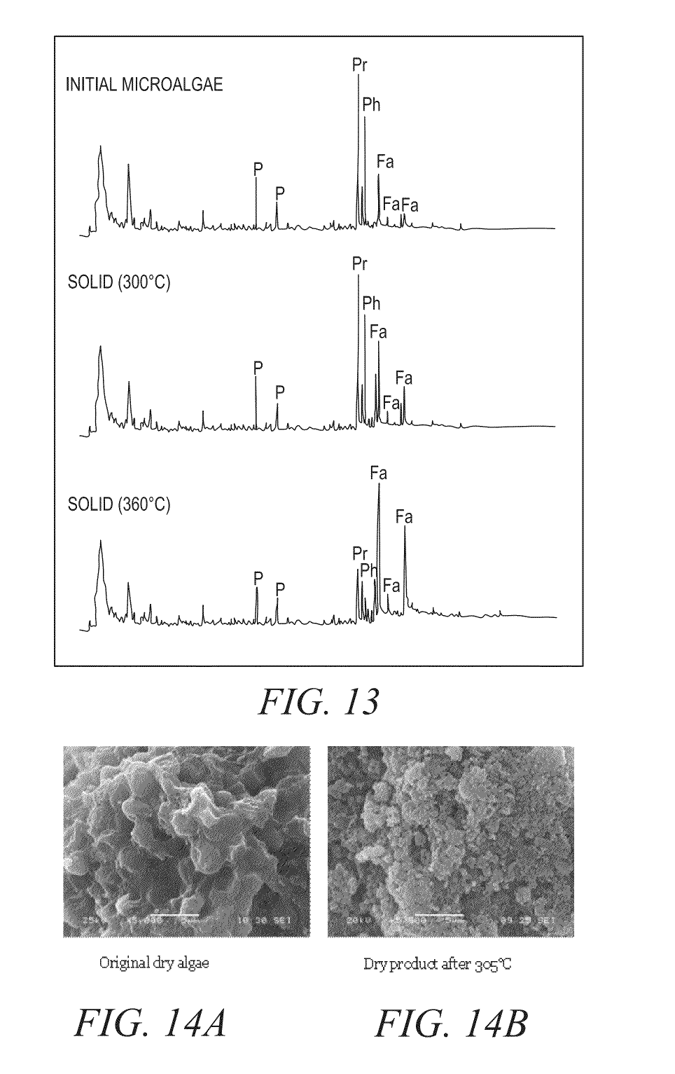

FIG. 13 displays the chromatograms obtained by pyr-GC-MS of the initial algae sample and both solid residues recovered at 300° C. and 360° C. Most of the peaks in the chromatograms have been assigned to isoprenoid hydrocarbons that likely derived from the lipid components of the algae. Fatty acids (Fa) are also detected and their relative proportion increases with temperature, which is consistent with the increase of hydrogen to carbon ratio. In both chromatograms of the solid residues, protein derived compounds have been identified and their proportion decreases with temperature. This shows that subcritical water extraction is able to deplete the residue of proteins. Characteristic biomarkers of chlorophyll, pristane (Pr) and phytane (Ph), are also detected and their presence explains the green color of the residues. Their proportions significantly decrease by increasing temperature.

The solid samples were further analyzed by scanning electron microscope (SEM) analysis (FIGS. 14A and B). The SEM images showed that the solids became smaller in size and appeared globular after the treatment. These particles seem to be formed due to the re-condensation process. It appears that most of the components were solubilized at the reaction conditions due to the increased solubility of organic matters in subcritical water. As the product cooled down, the unhydrolyzed fractions of lipids, carbohydrate, and proteins, along with macromolecule algaenan precipitated as globular particles due to difference in solubility in ambient water.

The flash hydrolysis in subcritical water to extract protein from microalgae as a previous step before conversion to biofuels can produce bioproducts from wet algae without the need of any chemical. The examples were focused on the extraction of proteins from Scenedesmus spp. using subcritical water in a continuous flow reactor, but it must be noted that the versatility of the setup allows working with any microalgae species slurry.

The quality of the protein extracted with the methods disclosed herein is high, as the chromatograms for the amino acid profiling of the analyzed samples show. The colorimetric protein estimation (Lowry's method) indicates that almost all the extracted protein is in the form of peptides and some free amino acids, and this is further confirmed by the ion chromatography analysis that reveal good amino acid profiles after treating the samples with diluted hydrochloric acid. Further, the relatively protein free solids after subcritical water extraction were characterized for biofuels production. About 60-80% of the total nitrogen content in the microalgae could be extracted within a few seconds of residence time in the range of 200-350° C. At the same time, lipids were retained in solids and their fraction increased from 7.0 wt % (untreated microalgae) to 71.8 wt % (treated at 305° C.) in subcritical water.

The disclosed processes are versatile and can be used for extracting nitrogen or proteins from most microalgae species, such as chlorella, nanochloropsis, Scenedesmus spp.

The disclosed processes are designed to extract nitrogen (up to 80%) in a few seconds (5 s to 60 s) by using subcritical water conditions in the temperature range of 200 to 360° C. without use of any catalyst.

The lipid component of microalgae is preserved during the flash hydrolysis step and, in fact, the treated solid becomes richer in lipids. Thus, the solids can be used for biofuels production with the reduced emission of NOₓ.

The processes provide the opportunity to recycle major nutrients (e.g. nitrogen, phosphorous, inorganics) and water to the algal pond and thus reduce the demand of nutrients during microalgae cultivation. Microalgae slurry with different solid contents can be used in the disclosed processes.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A method of fractionating proteins from algae comprising:
feeding to a reactor a biomass of algae saturated with water;
bringing the water saturating the algae to a subcritical temperature within the reactor; and
separating a reactor effluent into solids and liquid.

2. The method of claim 1, wherein feeding the biomass comprises:
providing algae from an algae source;
providing water from a water source; and
mixing the algae and water prior to feeding the reactor.

3. The method of claim 1, wherein the subcritical temperature is between 200° C. and 350° C.

4. The method of claim 1, wherein the reactor is a continuous feed reactor and a residence time of within the reactor is less than two minutes.

5. The method of claim 4, wherein the residence time is less than or equal to ten seconds.

6. The method of claim 1, wherein the algae saturated with water has a solids load of between 0.01 and 7.5 dry weight percent.

7. The method of claim 1 further comprising recycling the liquid for use in algae cultivation.

8. The method of claim 1 further comprising:
selecting the subcritical temperature in combination with a residence time to extract at least 60 percent of a total nitrogen content in the algae from the solids into the liquid.

9. The method of claim 8, wherein the nitrogen content of the liquid is in the form of proteins and peptides.

10. The method of claim 9, wherein the proteins and peptides are amino acids.

11. The method of claim 8, wherein the subcritical temperature is between 280° C. and 350° C.

12. The method of claim 1 further comprising:
selecting the subcritical temperature in combination with a residence time to optimize the lipid concentration in the solids.

13. The method of claim 12, wherein the subcritical temperature is between 200° C. and 300° C.

14. The method of claim 1 further comprising:
using the solids as a feedstock for biofuel.

15. A method of fractionating proteins from algae comprising:
feeding to a reactor an algal biomass;
providing a solvent consisting essentially of water to the reactor;
using the solvent as a reaction medium in the reactor by bringing the solvent to a subcritical temperature within the reactor; and
separating a reactor effluent into solids and liquid.

16. The method of claim 15, wherein feeding the algal biomass and providing the solvent occur in a single feed stream to the reactor.

17. The method of claim 15 further comprising:
selecting the subcritical temperature of the reaction medium to increase nitrogen extraction from the algal biomass to the liquid.

18. The method of claim 17, wherein the subcritical temperature is selected from between 280° C. and 350° C.

19. The method of claim 15 further comprising:
selecting the subcritical temperature of the reaction medium to increase lipid concentration in the solids.

20. The method of claim 19, wherein the subcritical temperature is selected from between 200° C. and 300° C.
